(19) United States
(12) Patent Application Publication
(10) Pub. No.: US 2023/0101832 A1
(43) Pub. Date: Mar. 30, 2023

(54) STABLE, LIQUID EMULSIFIERS ON THE BASIS OF CITRATE ESTERS AND THEIR USE

(71) Applicant: SYMRISE AG, Holzminden (DE)

(72) Inventors: Nikolas BUGDAHN, Holzminden (DE); Sabine LANGE, Holzminden (DE); Ricarda KRÄLING, Holzminden (DE); Vanessa SCHADE, Holzminden (DE)

(21) Appl. No.: 17/904,639

(22) PCT Filed: Feb. 25, 2020
(86) PCT No.: PCT/EP2020/054915

§ 371 Date: Aug. 19, 2022

(51) Int. Cl. A61K 31/225; A23L 35/00; A61K 9/107; A61K 8/365; A61K 8/37; A61K 8/06; C11D 3/20; C11D 17/00

(52) U.S. Cl. CPC A61K 31/225; A23L 35/10; A61K 9/107; A61K 8/365; A61K 8/375; A61K 8/062; C11D 3/2093; C11D 17/0017

(57) ABSTRACT

The present invention is in the field of emulsifiers and provides novel emulsifier mixtures for use in cosmetic or pharmaceutical formulations or in detergents. The emulsifier mixtures according to the invention comprise a citrate ester mixture and at least one 1,2-alkanediol, 1,3-alkanediol, 1,4-alkanediol and/or 1,5-alkanediol and are characterized by increased stability and low precipitation over a wide temperature range and during a long storage period.

### TECHNICAL FIELD

The present invention is in the field of emulsifiers and provides novel emulsifier mixtures for use in cosmetic or pharmaceutical formulations or in detergents. The emulsifier mixtures according to the invention comprise a citrate ester mixture and at least one 1,2-alkanediol, 1,3-alkanediol, 1,4-alkanediol and/or 1,5-alkanediol and are characterized by increased stability and low precipitation rate over a wide temperature range and during a long storage period.

### BACKGROUND OF THE INVENTION

Emulsifiers are very popular in cosmetics, pharmaceuticals and food production. Especially emulsifiers based on natural fats/oils and citric acid have been known for a long time. They are used in the food industry as emulsifiers, complexing agents (e.g. to support the effect of antioxidants) and carriers with the designation E472c (citric acid glycerol esters of mono- and diglycerides of fatty acids or citrem) and can be found among other things in cakes, cookies, puff pastries, bread, sausages, ice cream and desserts, confectionery and bakery fats (Schuster, G., et al., Emulsifiers for Food, Berlin, Heidelberg, New York, Tokyo: Springer 1985, pp. 107-114). Since the citric acid glycerol esters are based on natural oils, they also have fatty acid residues of different lengths corresponding to the natural oils. Depending on the oil used, these can have a fatty acid chain length of 8 to 18 C-atoms.

Emulsifiers are auxiliary agents for the production and stabilization of emulsions, which can be described as surface-active substances or surfactants in the narrower sense and are usually present as oily to waxy, but also powdery substances. Emulsifiers reduce the interfacial tension between the phases of emulsions and stabilize the emulsion formed. The structural characteristic of emulsifiers is their amphiphilic molecular structure. A molecule of such a compound has at least one group with affinity for substances of strong polarity and at least one group with affinity for non-polar substances. Emulsifiers can also be used, for example, as liquid emulsifier mixtures, the use of which is particularly advantageous in liquid cleaning agents or cream formulations sold as emulsions.

Emulsions are disperse systems of two or more immiscible liquids or immiscible liquid phases. One of the liquid phases forms the dispersion medium (also: outer, continuous or coherent phase), in which the other phase (also: inner or disperse phase) is distributed in the form of fine droplets. The particle diameter of the particles, which are preferably assumed to be spherical in an idealized view or whose size is given as the size of an equivalent sphere of the same diameter (“equivalent sphere”), varies. Most emulsions show non-uniform particle size and are polydisperse. Most natural and technical emulsions consist of water and oil or fat as immiscible phases. An O/W emulsion or oil-in-water emulsion is a fat-water mixture whose continuous phase is aqueous. Accordingly, an O/W emulsifier is an emulsifier that stabilizes an O/W emulsion or contributes to its stability. The oil phase of an O/W emulsion usually involves the use of a vegetable oil. Vegetable oils with a high content of unsaturated fatty acids tend to oxidize after a certain storage period. This causes oils to become “rancid”, which is accompanied by a marked reduction in odor.

Numerous citric acid glycerol esters, hereinafter referred to as citrate esters, are described in the prior art.

For example, Chinese patent application CN105541614A discloses emulsifiers that are citrate esters with a fatty acid chain length of 14, 16 and 18 C atoms. U.S. Pat. No. 4,071,544A discloses a process for the preparation of citrate esters with various mono- and diglycerides.

European patent application EP2111850A1 and U.S. Pat. No. 2,813,032A deal with the production of citrate esters from natural oil sources. Disclosed here are citrate esters based on sunflower oil or corn germ oil, which have a fatty acid chain length of 16 or 18 C atoms. The C16/C18 citrate esters described herein are commercially available citrate esters that are used as standard in various emulsions.

Apart from their use in liquid emulsions, citrate esters can also be used for other purposes. JP2012031250A and US20110273646A1 disclose citrate esters of caprylic acid, stearic acid and oleic acid as an ingredient for the preparation of protective films for optical polarizers, in particular optical polarizers in liquid crystal-based liquid crystal display (LCD) devices with the aim of providing LCD devices particularly well resistant to temperature and humidity.

Citrate esters have the advantage that their precursors can be obtained from natural oils and are therefore more environmentally friendly than artificially produced emulsifiers. Furthermore, citrate esters exhibit very good emulsifying properties and can stabilize emulsions. Due to their origin, citrate esters produced from vegetable lipids often have a high content of C16/C18 fatty acids. Interesting vegetable reactants, however, have a high content of short-chain C12/C14 fatty acids, which are still far less common in the field. For these, too, new processes for efficient esterification with citric acid must therefore be continuously made available in order to make them usable on the one hand, but also stable in the end-product.

A particular challenge in emulsifier production is the manufacture and preservation of liquid emulsifier mixtures. Although there are a large number of available emulsifiers based on citrate esters with different fatty acid chain lengths, it is evident that these liquid emulsifier mixtures become cloudy when exposed to temperature fluctuations and over a longer storage period. The resulting turbidity is due to the crystallization of the emulsifiers and is undesirable in the further use of these emulsifiers.

The primary task of the present invention is therefore to provide emulsifier mixtures, which possess the positive properties of citrate esters with a wide range of chain lengths of the respective fatty acid residues and at the same time remain stable over a wide temperature range, as well as over a long storage period and do not crystallize out.

Further tasks underlying the present invention result from the following explanations and the appended patent claims.

### SUMMARY OF THE INVENTION

The primary task of the present invention was solved by providing novel emulsifier mixtures comprising a citrate ester mixture and at least one 1,2-alkanediol, 1,3-alkanediol, 1,4-alkanediol and/or a 1,5-alkanediol, wherein the citrate ester mixture is a citric acid-glycerol ester mixture and comprises or consists of at least one compound of formula (I) and/or a salt thereof (for the meaning of R¹, R² and R³, see further below).

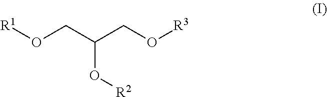

Furthermore, the present invention relates to the use of novel emulsifier mixtures for achieving reduced turbidity and precipitation in a liquid emulsifier mixture. In addition, the present invention relates to stable oil-in-water (O/W) emulsions comprising the emulsifier mixtures according to the invention and to preparations comprising the emulsifier mixtures according to the invention.

Further aspects of the present invention as well as particularly suitable embodiments will be apparent from the following description, examples and the appended patent claims.

### LIST OF FIGURES

FIG. 1 depicts photographic images of five different emulsifier mixtures with sunflower oil and 1,2-pentanediol corresponding to compositions of Table 2. Shown is a comparison between the solid, frozen emulsifier at −21° C. (start) and the emulsifier after thawing in liquid form at room temperature.

FIG. 2 depicts photographic images of three different emulsifier mixtures with caprylic capric triglyceride (INCI) and 1,2-pentanediol corresponding to the compositions of Table 3. Shown is a comparison between the solid, frozen emulsifier at −21° C. (start) and the emulsifier after thawing in liquid form at room temperature.

FIG. 3 depicts photographic images of two different emulsifier mixtures of a C16/C18 citrate ester market sample with caprylic capric triglyceride (INCI) and 1,2-pentanediol corresponding to the compositions of Table 4. Shown is a comparison between the solid frozen emulsifier at −21° C. (start) and the emulsifier after thawing in liquid form at room temperature.

FIG. 4 depicts photographic images of seven different emulsifier mixtures of C12/C14 citrate esters with 4-hydroyacetophenone and various 1,2-alkanediols corresponding to the compositions of Table 7. Shown is a comparison between the solid, frozen emulsifier at −21° C. (start) and the emulsifier after thawing in liquid form at room temperature.

FIG. 5a shows photographic images of six different emulsifier mixtures of C12/C14 citrate esters in combination with glyceryl caprylate and different concentrations of 1,2-pentylene glycol (a 1,2-alkanediol, namely 1,2-pentanediol) from Table 8 (experiments V2G as well as S1G-S5G). Shown is a comparison after one week of storage at ambient (room) temperature (see Example 5 below).

FIG. 5b shows photographic images of the emulsifier mixtures from Table 8 (experiments V2G as well as S1G-S5G) after thawing in liquid form at room temperature.

### DETAILED DESCRIPTION

In a first aspect of the present invention, the present invention relates to an emulsifier mixture comprising a citrate ester mixture and at least one 1,2-alkanediol, 1,3-alkanediol, 1,4-alkanediol and/or a 1,5-alkanediol, wherein the citrate ester mixture is a citric acid-glycerol-ester mixture, and wherein the citrate ester mixture comprises or consists of at least one compound of the following formula (I) and/or a salt thereof:

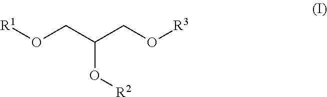

where in each case:

(i) at least one of R¹, R² and R³ is a citric acid residue attached to the glycerol backbone through an ester bond;

(ii) at least one of R¹, R² and R³ is a fatty acid residue attached to the glycerol backbone by an ester bond;

and whereby

the proportion of the total amount of the compound of formula (I) comprising at least one fatty acid residue independently selected from the group consisting of the fatty acid residue of caproic acid, the fatty acid residue of caprylic acid, the fatty acid residue of capric acid, the fatty acid residue of lauric acid and the fatty acid residue of myristic acid is more than 55 wt.-%, preferably more than 70 wt.-% or more than 75% wt.-%, particularly preferably more than 90 wt.-%, based on the total weight of the compounds of the formula (I);

and/or

wherein the proportion of the total amount of the compound of formula (I) comprising at least one fatty acid residue independently selected from the group consisting of the fatty acid residue of lauric acid and the fatty acid residue of myristic acid is more than 50 wt.-%, preferably more than 55 wt.-%, more preferably more than 60 wt.-%, based on the total weight of the compounds of formula (I);

and/or

wherein the proportion of the total amount of the compound of formula (I) comprising at least one fatty acid residue independently selected from the group consisting of the fatty acid residue of palmitic acid, the fatty acid residue of palmitoleic acid, the fatty acid residue of stearic acid, the fatty acid residue of oleic acid, the fatty acid residue of linoleic acid and the fatty acid residue of linolenic acid is less than 40 wt.-%, preferably less than 35 wt.-%, particularly preferably less than 30 wt.-%, based on the total weight of the compounds of formula (I).

The compounds of formula (I) to be used according to the invention have an amphiphilic molecular structure.

In the context of the present invention, “citric acid” or the “citric acid residue” derived therefrom means citric acid (2-hydroxypropane-1,2,3-tricarboxylic acid, in particular CAS: 77-92-9 or InChIKey: KRKNYBCHXYNGOX-UHFFFAOYSA-N) or the residue derived therefrom as well as their diastereomers or the residues derived therefrom and their enantiomers or the residues derived therefrom, in particular isocitric acid (3-carboxy-2-hydroxy-pentane-1,5-diacid, in particular InChIKey: ODBLHEXUDAPZAU-FONMRSAGSA-N) or the radical derived therefrom and the enantiomers or the residues derived therefrom.

A citric acid residue bound by an ester bond is to be understood according to the present invention as a structural unit to which one of the following formulae (iii-a) or (iii-b), applies:

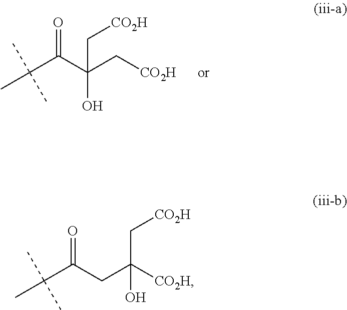

wherein the dashed line marks the bond linking each of the radicals R¹, R² or R³ independently of one another to each of the oxygen atoms denoted by “O” in the compounds of formula (I), taking into account the preceding explanations and requirements with respect to the compounds of formula (I).

A “fatty acid residue” in the sense of the present invention is to be understood as a structural component for which the following formula applies:

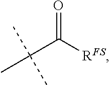

wherein the dotted line marks the bond which independently links one of the radicals R¹, R² or R³ with respect to the compounds of the formula (I) to one of the oxygen atoms designated “O” in the compounds of the formula (I) and wherein R^{FS} is a univalent radical selected from the group consisting of alkyl radicals, alkadienyl radicals and alkatrienyl radicals in accordance with the nomenclature known to the skilled person.

For the purposes of the present invention, “short-chain fatty acid residues” are preferably residues of the fatty acids as defined below. Preferably, short-chain fatty acid residues are residues selected from the group consisting of the corresponding residues of fatty acids having 6 to 14 carbon atoms, in particular caproic acid, caprylic acid, capric acid, lauric acid and myristic acid. Particularly preferred are short-chain fatty acid residues selected from the group consisting of the associated residues of fatty acids having 12 to 14 carbon atoms, in particular lauric acid and myristic acid. “Long-chain fatty acid residues” in the sense of the present invention are preferably residues of the fatty acids selected from the group consisting of the associated residues of the fatty acids having more than 14 carbon atoms, in particular palmitic acid, palmitoleic acid, stearic acid, oleic acid and linolenic acid. The structural formulae of the fatty acid residues to be used according to the invention are shown below:

the fatty acid residue of caproic acid having the formula (ii-a):

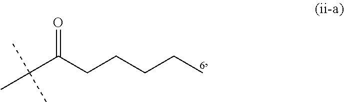

the fatty acid residue of caprylic acid having the formula (ii-b):

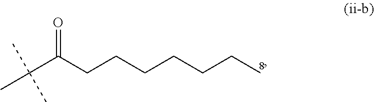

the fatty acid residue of capric acid having the formula (ii-c):

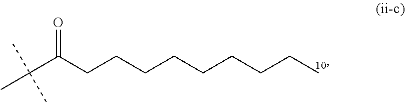

the fatty acid residue of lauric acid having the formula (ii-d):

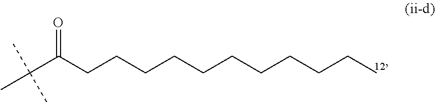

the fatty acid residue of myristic acid having the formula (ii-e):

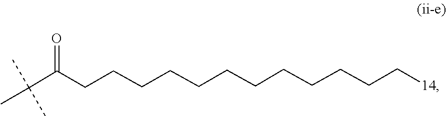

the fatty acid residue of palmitic acid having the formula (ii-f):

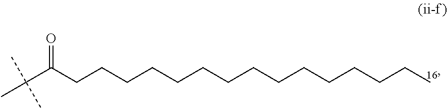

the fatty acid residue of palmitoleic acid having the formula (ii-g):

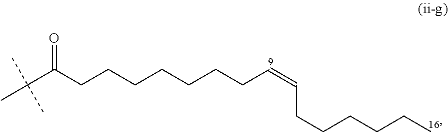

the fatty acid residue of stearic acid having the formula (ii-h)

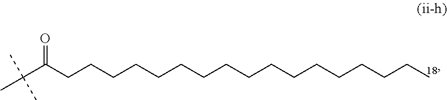

the fatty acid residue of oleic acid having the formula (ii-i):

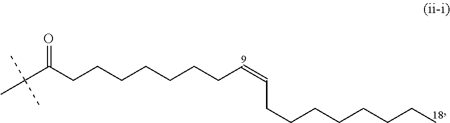

the fatty acid residue of linoleic acid with the formula (ii-j):

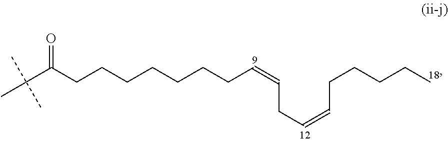

and the fatty acid residue of linolenic acid with the formula (ii-k):

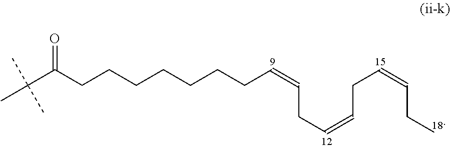

In the event of any discrepancies between a structural formula shown and the name given for the compound or the respective structural element, or if the name does not fully comply with the convention, the respective structural formula given or the respective structural element applies. Whenever the abbreviations C8, C10, C12, C14, C16 or C18 are used in the context of the present invention, they refer to the number of carbon atoms of the fatty acid residues.

An “alkanediol” within the meaning of the present invention is a compound which consists of straight or branched hydrocarbon chains and contains exactly two hydroxy groups at different positions. The hydroxy groups are attached to different carbon atoms, resulting in the nomenclatures 1,2-alkanediol, 1,3-alkanediol, 1,4-alkanediol, and so on. In the case of a 1,2-alkanediol, the hydroxyl groups are bonded to the first and second carbon atoms, and in the case of a 1,3-alkanediol to the first and third alkanediols. The same applies analogously to the 1,4-alkanediols and 1,5-alkanediols of the invention.

The fatty acid residues (ii-a) to (ii-k) described above as constituents, thus one or more of the residues R¹, R² and R³, of the emulsifiers according to the invention exhibit advantageous solubilizing properties over or stronger emulsifying action than emulsifiers known in the prior art and are also suitable for use in a broader range of applications than prior art emulsifiers. Surprisingly, it was found that the citric acid esters according to the invention can be advantageously used together with at least one 1,2-, 1,3-, 1,4- or 1,5-alkanediol in liquid emulsifier mixtures. The mixture according to the invention thereby shows improved stability over a wide temperature range, low crystallization of the citric acid esters during storage and improved odor of the emulsifier mixture during storage.

The proportion of the total amount of compounds of the formula (I) and salts of the compounds of the formula (I) relative to the total weight of the emulsifier is preferably determined by comparison of mass spectra with reference spectra, taking into account the elution position and the UV spectra. High-resolution time-of-flight (TOF) mass spectra can be used to determine the molar mass of the compound and, if necessary, a molecular formula. The sample is separated by liquid chromatography and detected by mass spectrometry (MS). The mass spectrum generated allows identification of the individual components. It is possible to generate an MS spectrum from the substance for further identification, as well as to perform detection via light or laser light scattering. Alternatively, gravimetric analysis is also applicable. Minor deviations in the determination of the wt % data, e.g. due to different measuring methods, are acceptable within the scope of the present invention and are not relevant for the practicability of the objects of the invention described herein.

Another embodiment of the present invention relates to an emulsifier mixture according to the invention, wherein the 1,2-, 1,3-, 1,4-, or the 1,5-alkanediol is independently selected from the group consisting of a corresponding C5-C12 alkanediol, preferably wherein the alkanediol is selected from 1,2-pentanediol, 1,2-hexanediol, 1,2-heptanediol, 1,2-octanediol, 1,2-nonanediol, 1,2-decanediol, 1,2-dodecanediol, 1,3-propanediol, 1,4-butanediol, or 1,5-pentanediol, or mixtures thereof.

Yet another embodiment of the present invention relates to an emulsifier mixture according to the invention, wherein the proportion of the citrate ester mixture is 10-98 wt.-%, preferably 30-80 wt.-%, and particularly preferably 40-70 wt.-%, and the proportion of the at least one 1,2-, 1,3-, 1,4- or 1,5-alkanediol is 1-90 wt.-%, preferably 2-75% wt.-%, and particularly preferably 4-50 wt.-%, in each case based on the total weight of the emulsifier mixture.

In the context of the present invention, it has been found to be particularly advantageous that a proportion of 2-75 wt.-% of 1,2-, 1,3-, 1,4- or 1,5-alkanediol is sufficient to obtain a stable emulsifier mixture. It is particularly advantageous to use 4-50 wt.-% of alkanediol in the emulsifier mixture according to the invention, since a stable emulsifier mixture could already be obtained with this amount.

According to one embodiment, the present invention relates to an emulsifier mixture according to the invention, wherein the emulsifier mixture additionally comprises 1-20 wt.-%, preferably 2-10 wt.-%, and particularly preferably 3-9 wt.-%, preferably 5-8 wt.-% of 4-hydroxyacetophenone, preferably wherein the emulsifier mixture comprises 4-8 wt.-% 4-hydroxyacetophenone in combination with 4-30 wt.-%, preferably 5-10 wt.-% of at least one 1,2-alkanediol, wherein the 1,2-alkanediol is preferably 1,2-octanediol, or a mixture of 1,2-alkanediols comprising at least 0.5-5 wt.-% 1,2-octanediol.

4-Hydroxyacetophenone is a phenolic component found mainly in the roots of Norwegian pine trees. It can be used in pharmacy in various medicines. In cosmetics, this component is mainly used as a fragrance. In the context of the present invention, it was surprisingly found that 4-hydroxyacetophenone is capable of synergistically interacting with the alkanediols according to the invention and thereby having a direct influence on the stability of the emulsifier mixture according to the invention. As a result, particularly stable emulsifier mixtures can be obtained by admixture, which are practicable and cost-efficient in production. Emulsifier mixtures comprising a combination of 4-hydroxyacetophenone and 1,2-octanediol or a mixture of alkanediols with 1,2-octanediol are particularly preferred.

In yet another embodiment, the present invention relates to an emulsifier mixture according to the invention, wherein the at least one fatty acid residue of the citrate ester mixture is obtained from a fatty acid-containing reactant, wherein the highest fatty acid content of the reactant in % by weight based on the total fatty acid content is on C8 to C18 fatty acids, preferably on C8 to C14 fatty acids, particularly preferably on C12 to C14 fatty acids.

Another embodiment of the present invention relates to an emulsifier mixture according to the invention, wherein the at least one fatty acid residue of the citrate ester mixture is of natural, biotechnological or chemical origin.

In the context of the present invention, a fatty acid residue of natural origin is a fatty acid residue obtained from natural sources, such as oils, plant extracts or plant seeds. A fatty acid residue of biotechnological origin describes fatty acid residues obtained by fermentative conversion using microorganisms, fungi, plant cells or mammalian cells. The term “fatty acid residue of chemical origin” describes a fatty acid residue obtained by chemical catalysis.

In yet another embodiment, the present invention relates to an emulsifier mixture according to the invention, wherein the fatty acid-containing reactant is selected from the group consisting of coconut oil, babassu oil, sunflower oil, rapeseed oil, neutral oil, palm kernel oil, macúba oil, microalgae oil, and mixtures thereof.

According to a further embodiment, the present invention relates to an emulsifier mixture according to the invention, wherein the emulsifier mixture is a liquid and optionally comprises at least one lipophilic solvent.

A liquid emulsifier mixture is particularly preferred in the context of the present invention because it can be used directly in a wide range of formulations, without prior dissolution and further formulation steps. This is particularly advantageous in liquid or cream formulations where the final product is a homogeneous mixture. Surprisingly, it was shown that the emulsifier mixture according to the invention is preferably present as a liquid emulsifier mixture and exhibits increased stability and reduced crystallization over a wide temperature range, as well as over a long storage period.

A “lipophilic solvent” in the context of the present invention refers to a solvent in which fats and oils dissolve or which itself can dissolve fats and oils well. Examples include fats, as well as oils of natural, biotechnological or chemical origin.

Another embodiment of the present invention relates to an emulsifier mixture according to the invention, wherein the lipophilic solvent comprises 5-60 wt. % (w/w) of the emulsifier mixture and is selected from the group consisting of coconut oil, babassu oil, sunflower oil, rapeseed oil, such as propanediol dicaprylate caprate (INCI), cetearyl nonanoate (INCI), Diisopropyl Adipate (INCI), Glyceryl Caprylate (INCI), 20 Ethylhexyl Stearate (INCI), Triethyl Citrate (INCI), Polyglyceryl 4 Caprate (INCI), Polyglyceryl 3 Caprate (INCI), Microalgae Oil and mixtures thereof.

Whenever the present invention refers to “INCI”, it refers to the “International Nomenclature for Cosmetic Ingredients” as part of EC Regulation No. 1223/2009 (EU Cosmetics Regulation). Therefore, for ease of understanding of the relevant terminology, the present disclosure uses the English, but common in the field, INCI term where appropriate, as a translation could potentially lead to ambiguity.

A further embodiment of the present invention relates to an emulsifier mixture according to the invention, wherein the emulsifier mixture has a reduced oxidation and thus an extended shelf life, wherein the extended shelf life is at least one month, preferably at least three months, and particularly preferably at least six months relative to an emulsifier mixture not according to the invention.

As described above, vegetable oils with a high content of unsaturated fatty acids tend to oxidize after a certain storage period. This causes oils to become “rancid,” which is accompanied by a marked reduction in odor. Surprisingly, it was shown that the emulsifier mixture according to the invention exhibits reduced oxidation of the lipophilic solvent used. In addition to the aspects already described above, this leads to an extended shelf life and stabilization of the emulsifier mixture according to the invention.

Another aspect of the present invention relates to the use of an emulsifier mixture according to the invention for achieving reduced turbidity and precipitation at temperatures between about −20° C. to +45° C., preferably between about 5-25° C., in a liquid emulsifier mixture according to the invention, wherein the liquid emulsifier mixture preferably exhibits a homogeneous phase transition upon thawing, or remains a clear solution after storage.

One embodiment of the present invention relates to the use of a liquid emulsifier mixture comprising a citrate ester mixture, wherein the citrate ester of the citrate ester mixture comprises at least one fatty acid residue independently selected from the group consisting of the fatty acid residue of palmitic acid, the fatty acid residue of palmitoleic acid, the fatty acid residue of stearic acid, the fatty acid residue of oleic acid, the fatty acid residue of linoleic acid and the fatty acid residue of linolenic acid, and wherein the emulsifier mixture comprises at least one 1,2-alkanediol, 1,3-alkanediol, 1,4-alkanediol and/or a 1,5-alkanediol, to achieve reduced turbidity and precipitation at temperatures between about −20° C. to +45° C., preferably between about 5-25° C., wherein the liquid emulsifier mixture preferably exhibits a homogeneous phase transition upon thawing, or remains a clear solution after storage.

A “reduced” turbidity in the context of the present invention refers to the increased optical light transmission of an emulsifier mixture according to the invention compared to an emulsifier mixture not according to the invention. Suitable methods for determining turbidity are sufficiently known to the person skilled in the art.

The turbidity of an emulsifier mixture is directly associated with the proportion of crystals in the emulsifier mixture. The more crystals present in an emulsifier mixture, the more turbid and consequently the more inhomogeneous this mixture is. It is therefore advantageous in the context of the present invention for the emulsifier mixture to have little or no turbidity. “Precipitation” in the context of the present invention refers to crystallization of the citrate esters in a mixture. Turbidity is directly associated with precipitation of the citrate esters and it is advantageous if this is prevented, since precipitation leads to inhomogeneity of a mixture. Emulsifier mixtures are particularly susceptible to phase transition, such as occurs during frozen storage and subsequent thawing of the emulsifier. Emulsifier mixtures not according to the invention show turbidity after thawing, which is associated with precipitation of the citrate esters. Surprisingly, it was found that the emulsifier mixtures according to the invention show slight to no turbidity as well as a homogeneous phase transition.

Although short-chain fatty acid residues are preferred in the context of the present invention, it was surprisingly found that emulsifier mixtures having citrate esters with long-chain, commercially available fatty acid residues (C16/C18) are also stable over a long storage period and no turbidity is observed. Thus, the emulsifier mixtures according to the invention offer an advantage over commercially available emulsifier mixtures not according to the invention over a wide range of fatty acid residue lengths. Furthermore, the disclosed mixtures allow a wide range of natural reactants to be used without problems, regardless of the respective fatty acid composition of the natural product.

According to a further embodiment, the present invention relates to a use according to the invention for odor improvement, preferably for odor improvement of a liquid emulsifier mixture, preferably a liquid emulsifier mixture as described above.

In yet another embodiment, the present invention relates to a use of an emulsifier mixture, preferably a liquid emulsifier mixture, comprising a citrate ester mixture, wherein the citrate ester of the citrate ester mixture comprises at least one fatty acid residue independently selected from the group consisting of the fatty acid residue of palmitic acid, the fatty acid residue of palmitoleic acid, the fatty acid residue of stearic acid, the fatty acid residue of oleic acid, the fatty acid residue of linoleic acid, and the fatty acid residue of linolenic acid, and wherein the emulsifier mixture comprises at least one of a 1,2-alkanediol, 1,3-alkanediol, 1,4-alkanediol, and a 1,5-alkanediol, for odor improvement.

Liquid emulsifier mixtures tend to have an unpleasant intrinsic odor, so an odor improvement, i.e. a subjectively improved odor perception, is desirable. Especially when liquid emulsifier mixtures are used in care products, a strong intrinsic odor is disadvantageous, as this reduces consumer acceptance.

In yet another embodiment, the present invention relates to a use according to the invention of an emulsifier mixture according to the invention or a liquid emulsifier mixture according to the invention for improved emulsifiability and/or stability of an oil-in-water emulsion.

An improved emulsifiability is characterized by the faster attainment of a uniform emulsion in which two phases are no longer recognizable. The stability of an emulsion refers to the condition that the emulsion does not separate into two phases and remains visually recognizable as one phase.

Another aspect of the present invention relates to an emulsion comprising an oil phase containing at least one emulsifier mixture according to the invention or at least one liquid emulsifier mixture according to the invention or a mixture thereof and an aqueous phase and optionally further comprising at least one compound for lowering or increasing the viscosity of the emulsion, wherein the emulsion is an oil-in-water emulsion.

The components of the oil phase of emulsions according to the invention can advantageously be selected from the group of branched and unbranched hydrocarbons and hydrocarbon waxes, silicone oils, dialkyl ethers, the group of saturated or unsaturated, branched or unbranched alcohols, and of fatty acid triglycerides, namely triglycerol esters of saturated and/or unsaturated, branched and/or unbranched alkanecarboxylic acids having a chain length of 8 to 24, in particular 12 to 18, carbon atoms. The fatty acid triglycerides can advantageously be selected, for example, from the group of synthetic, semisynthetic and natural oils, e.g. olive oil, sunflower oil, soybean oil, peanut oil, rapeseed oil, almond oil, palm oil, coconut oil, babassu oil, microalgae oil and palm kernel oil.

The aqueous phase of emulsions according to the invention optionally advantageously contains water-soluble plant extracts, alcohols, diols or polyols (lower alkyl), as well as their ethers, preferably ethanol, isopropanol, propylene glycol, glycerol, ethylene glycol monoethyl or monobutyl ether, propylene glycol monomethyl, monoethyl or monobutyl ether, diethylene glycol monomethyl or monoethyl ether and analogous products, alcohols, e. g.e.g. ethanol, 1,2-propanediol or glycerol.

Preferred in the sense of the present invention is an emulsion according to the invention, further comprising one or more different compound(s) for lowering or increasing the viscosity of the emulsion.

Viscosity is the property, especially of a liquid, of resisting the mutual laminar displacement of two adjacent layers. Accordingly, viscosity can also be understood as toughness or internal friction. For the specific adjustment of viscosity and the achievement of a defined consistency of emulsions or preparations, these can contain, for example, thickeners on an organic (alginate, tragacanth, xanthan, modified celluloses, carrageenans, etc.) and/or inorganic (bentonite, pyrogenic silica, magnesium aluminum silicates, etc.) basis.

Preferably, an emulsion according to the invention comprises one or more thickening agents which may advantageously be selected from the group consisting of silica, aluminosilicates, polysaccharides or their derivatives, e.g. hyaluronic acid, xanthan, hydroxyropylmethylcellulose, carbomer (Ultrez-10), in each case individually or in combination.

Yet another aspect of the present invention relates to a semi-finished product preparation, preferably a preparation serving for cleaning, a cosmetic or pharmaceutical, preferably a dermatological, preparation, a preparation serving for consumption or nutrition, comprising at least one emulsifier mixture according to the invention and/or at least one emulsion according to the invention.

“Semi-finished product preparation” in the sense of the present invention means products that are not completely finished, such as emulsions or preparations, which are further processed into finished products at a later stage. In this one or more final processing steps, further essential compounds may be added to the semi-finished product, dilution steps may take place, or the semi-finished product may be subjected to various mechanical processes in order to achieve certain macroscopic properties. A cosmetic or pharmaceutical, preferably dermatological, preparation serving cleansing purposes within the meaning of the present invention is preferably a preparation which, among other things, preferably serves cosmetic skin care purposes. Cosmetic skin care is primarily to be understood as strengthening or restoring the natural function of the skin as a barrier against environmental influences (e.g., dirt, chemicals, microorganisms) and against the loss of endogenous substances (e.g., water, natural fats, electrolytes), as well as supporting its corneal layer in its natural regenerative capacity when damage occurs. If the barrier properties of the skin are disturbed, this can lead to increased absorption of toxic or allergenic substances or to infestation by microorganisms and, as a result, to toxic or allergic skin reactions. The aim of skin care is also to compensate for the loss of oil and water from the skin caused by daily washing. This is particularly important when the natural regenerative capacity is insufficient. In addition, skin care products should protect against environmental influences, especially sun and wind, and delay skin aging.

Preferably, pharmaceutical preparations within the meaning of the invention are understood to be preparations which are available, for example, in the form of capsules, tablets (uncoated as well as coated tablets, e.g. enteric coatings), lozenges, granules, pellets, solid mixtures, dispersions in liquid phases, as emulsions, as powders, as solutions, as pastes or as other preparations which can be swallowed or chewed, and which are used as medicaments available on prescription, in pharmacies or otherwise, or as food supplements.

Medical topical compositions, as further examples of pharmaceutical preparations, generally contain one or more drugs in effective concentrations. For the sake of simplicity, reference is made to the legal provisions of the Federal Republic of Germany (e.g. Cosmetics Regulation, Food and Drug Act) to distinguish between cosmetic and medicinal applications and corresponding products.

The cosmetic and pharmaceutical preparations within the meaning of the present invention may contain excipients such as are commonly used in such preparations, e.g. Preservatives, antioxidants, vitamins, bactericides, perfumes, substances to prevent foaming, dyes, pigments having a coloring effect, thickeners, surfactants, emollients, emulsifiers, moisturizing and/or humectant substances, moisturizers, fats, oils, waxes, plant extracts or other common ingredients such as alcohols, lower alkyl alcohols, polyols, lower alkyl polyols, polymers, foam stabilizers, complexing agents, electrolytes, organic solvents, propellants, silicones or silicone derivatives.

Food or drink preparations in the sense of the present invention are e.g. bakery products (e.g. bread, dry cookies, cakes, other pastries), confectionery (e.g. chocolates, chocolate bar products, other bar products, fruit gums, hard and soft caramels, chewing gum), alcoholic or non-alcoholic beverages (e.g.E.g., coffee, tea, wine, wine-based beverages, beer, beer-based beverages, liquors, spirits, brandies, fruit-based sodas, isotonic beverages, soft drinks, nectars, fruit and vegetable juices, fruit or vegetable juice preparations), instant beverages (e.g.e.g., instant cocoa drinks, instant tea drinks, instant coffee drinks, instant fruit drinks), meat products (e.g., ham, fresh or raw sausage preparations, seasoned or marinated fresh or cured meat products), eggs or egg products (dried egg, egg white, egg yolk), cereal products (e.g., breakfast cereals, cereal bars, pre-cooked ready-to-eat rice products), dairy products (e.g. milk drinks, buttermilk drinks, milk ice cream, yogurt, kefir, cream cheese, soft cheese, hard cheese, dried milk powder, whey, butter, buttermilk, partially or wholly hydrolyzed milk protein-containing products), products made from soy protein or other soybean fractions (e.g. Soy milk and products made therefrom, fruit drinks containing soy protein, preparations containing soy lecithin, fermented products such as tofu or tempeh or products made therefrom), fruit preparations (e.g., jams, fruit ice cream, fruit sauces, fruit fillings), vegetable preparations (e.g., ketchup, sauces), and products containing soy protein. (e.g. ketchup, sauces, dried vegetables, frozen vegetables, pre-cooked vegetables, cooked vegetables), snacks (e.g. baked or deep-fried potato chips or potato dough products, corn- or peanut-based extrudates), fat- and oil-based products or emulsions thereof (e.g. mayonnaise, remoulade), and fat- and oil-based products. (e.g. mayonnaise, tartar sauce, dressings), other ready meals and soups (e.g. dry soups, instant soups, pre-cooked soups), spices, seasoning mixtures and in particular seasonings, which are used for example in the snack sector. The preparations in the sense of the invention can also serve as semi-finished goods for the production of further preparations serving nutrition or enjoyment. The preparations within the meaning of the invention may also be in the form of capsules, tablets (uncoated as well as coated tablets, e.g. enteric coatings), lozenges, granules, pellets, solid mixtures, dispersions in liquid phases, as emulsions, as powders, as solutions, as pastes or as other preparations which can be swallowed or chewed as food supplements.

Another aspect of the present invention relates to a preparation comprising at least one semi-finished product preparation according to the invention, wherein the preparation is preferably selected from the group consisting of a cleaning preparation, a cosmetic or pharmaceutical preparation, preferably a dermatological preparation, and an edible or nutritional preparation as defined above.

The present invention is explained in more detail below with reference to selected examples, although the present invention is not limited thereto.

### EXAMPLES

Whenever the w/w % term is used in the following examples, it refers to percent by weight.

### Example 1: Preparation of Emulsifier Mixtures with 1,2-Pentanediol

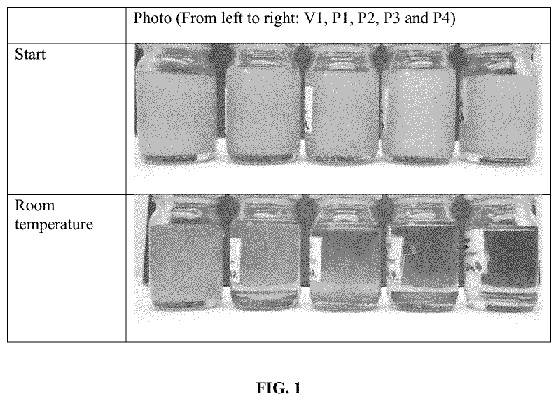

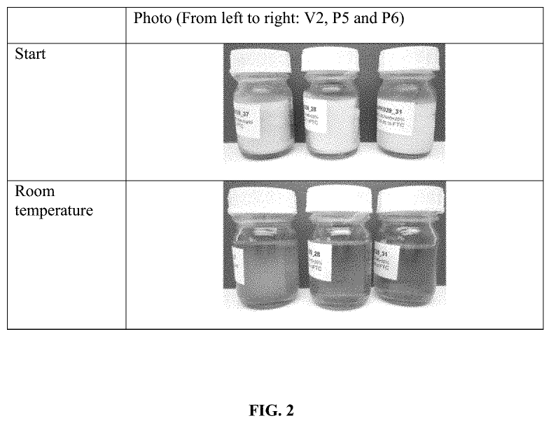

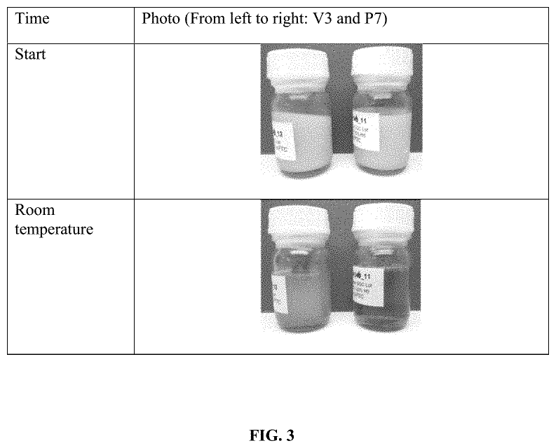

C12, C14, C16 or C18 citrate esters were heated to 80° C. while stirring. A lipophilic solvent (oil) and 1,2-pentanediol were added while stirring with a blade stirrer at 450 rpm. The obtained mixtures were frozen at −21° C. and then thawed at room temperature (about 23° C.-26° C.), photographic documentation was performed at −21° C. and room temperature. Evaluation of the mixtures was performed using the evaluation key in Table 1. The respective mixtures as well as the evaluation are described in Table 2, Table 3, and Table 4 and shown in FIGS. 1, 2 and 3.

TABLE 1
Evaluation key

| | Precipitation | Turbidity | Smell |
|---|---|---|---|
| Without findings | K (Clear) | K (Clear) | N |
| Easy | A | T | O |
| Significantly | A+ | T+ | O+ |
| Very strong | A++ | T++ | O++ |

TABLE 2
Formulations with 1,2-pentanediol and evaluation
w/w %

| | Comparative | | | | |
|---|---|---|---|---|---|
| | sample V1 | P1 | P2 | P3 | P4 |
| 1,2-pentanediol | 0 | 5 | 10 | 15 | 20 |
| Sunflower oil | 50 | 45 | 40 | 35 | 30 |
| C12/C14 citrate ester | 50 | 50 | 50 | 50 | 50 |
| Appearance at | T++ | T+ | T | K | K |
| Room temperature | A++ | | | | |

TABLE 3
Formulations with 1,2-pentanediol and evaluation
w/w %

| | Comparative | | |
|---|---|---|---|
| | sample V2 | P5 | P6 |
| 1,2-pentanediol | 0 | 10 | 20 |
| Caprylic Capric Triglyceride | 40 | 30 | 20 |
| (INCI) | | | |
| C12/C14 citrate ester | 60 | 60 | 60 |
| Appearance at Room temperature | T++ | T+ | T |
| | A++ | | |

TABLE 4
Formulations with 1,2-pentanediol, C16/C18 citrate esters and evaluation
w/w %

| | Comparative | |
|---|---|---|
| | sample V3 | P7 |
| C16/C18 citrate ester | 100 | 80 |
| Market pattern, containing 15% | | |
| Caprylic Capric Triglyceride | | |
| (INCI) | | |
| 1,2-pentanediol | — | 20 |
| Appearance at room temperature | T+ | K |
| | A+ | |

### Example 2: Preparation of Emulsifier Mixtures with 1,2-Hexanediol

C12, C14, C16 or C18 citrate esters were heated to 80° C. while stirring. A lipophilic solvent (oil) and 1,2-hexanediol were added while stirring with a blade stirrer at 450 rpm. The obtained mixtures were frozen at −21° C. and then thawed at room temperature (about 23° C.-26° C.), photographic documentation was performed at −21° C. and room temperature. Evaluation of the mixtures was performed using the evaluation key in Table 1. The formulation of the respective mixtures as well as the evaluation are given in

Table 5 shown.

TABLE 5
Formulations with 1,2-hexanediol and evaluation
w/w %

| | Comparative | | | Comparative | | |
|---|---|---|---|---|---|---|
| | sample V4 | H1 | H2 | sample V2 | H3 | H4 |
| 1,2-hexanediol | 0 | 10 | 20 | 0 | 10 | 20 |
| Sunflower oil | 40 | 30 | 20 | — | — | — |
| Caprylic Capric | — | — | — | 40 | 30 | 20 |
| Triglyceride | | | | | | |
| C12/C14 citrate | 60 | 60 | 60 | 60 | 60 | 60 |
| ester | | | | | | |
| Appearance at | T+ | T | T | T+ | T | T |
| room | A+ | | | A+ | | |
| temperature | | | | | | |

### Example 3: Mixtures with 1,2 Octanediol

C12, C14, C16 or C18 citrate esters were heated to 80° C. while stirring. A lipophilic solvent (oil) and 1,2-octanediol were added while stirring with a blade stirrer at 450 rpm. The obtained mixtures were frozen at −21° C. and then thawed at room temperature (about 23° C.-26° C.), photographic documentation was performed at −21° C. and room temperature. Evaluation of the mixtures was performed using the evaluation key in Table 1. The formulation of the mixtures as well as the evaluation is shown in Table 6.

TABLE 6
Formulations with 1,2-octanediol and evaluation
w/w %

| | Comparative | | |
|---|---|---|---|
| | sample V2 | O1 | O2 |
| 1,2-octanediol | 0 | 10 | 20 |
| Caprylic Capric Triglyceride | 40 | 30 | 20 |
| C12/C14 citrate ester | 60 | 60 | 60 |
| Appearance at room temperature | T+ | T | T |

### Example 4: Mixtures with Addition of 4-Hydroxyacetophenone

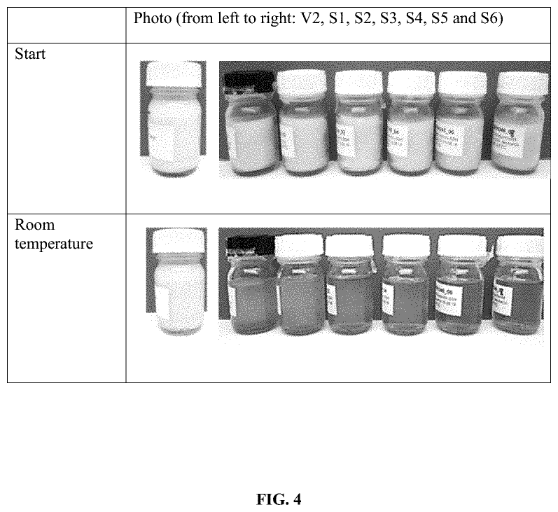

C12/C14 citrate esters were heated to 80° C. while stirring. A lipophilic solvent (oil), 1,2-alkanediol, and 4-hydroxyacetophenone were added while stirring with a blade stirrer at 450 rpm. The obtained mixtures were frozen at −21° C. and then thawed at room temperature (about 23° C.-26° C.), photographic documentation was performed at −21° C. and room temperature. Evaluation of the mixtures was performed using the evaluation key in Table 1. The formulation of the mixtures as well as the evaluation is presented in Table 7, the corresponding photographic images are shown in FIG. 4.

TABLE 7
Formulations with 4-hydroxyacetophenone and evaluation
w/w %

| | Comparative | | | | | |
|---|---|---|---|---|---|---|
| | sample V2 | S1 | S2 | S3 | S4 | S5 |
| C12/C14 citrate ester | 60 | 60 | 60 | 60 | 60 | 60 |
| Caprylic Capric | 40 | 35 | 30 | 30 | 30 | 30 |
| Triglyceride (INCI) | | | | | | |
| 4- | | — | 5 | 5 | 5 | 5 |
| hydroxyacetophenone | | | | | | |
| 1,2-pentanediol | — | 5 | 5 | — | — | — |
| 1,2-hexanediol | — | — | — | 5 | — | — |
| 1,2-octanediol | — | — | — | — | 5 | — |
| 1,2-hexanediol + | — | — | — | — | — | 5 |
| 1,2-octanediol | | | | | | |
| Appearance at room | T+ | T+ | T | T | K | K |
| temperature | A+ | | | | | |

### Example 5: Mixtures without Additional Lipid Phase

Another preferred formulation describes mixtures of the C12/C14 citrate ester with glyceryl esters with and without 1,2-pentanediol which are also, but not exclusively, suitable for surfactant formulation (e.g. rinse-off).

TABLE 8
Formulations with Glyceryl Caprylate

| | w/w % | | | | | |
|---|---|---|---|---|---|---|
| INCI | V2G | S1G | S2G | S3G | S4G | S5G |
| C12/C14 citrate | 60 | 60 | 60 | 50 | 50 | 50 |
| ester | | | | | | |
| Glyceryl Caprylate | 40 | 20 | 10 | 30 | 20 | 25 |
| (Symlite G8) | | | | | | |
| 1,2-pentanediol | — | 20 | 30 | 20 | 30 | 25 |
| Appearance after | T+ | K | K | K | K | K |
| 1 week storage | | | | | | |
| at room | | | | | | |
| temperature (22° C.) | | | | | | |

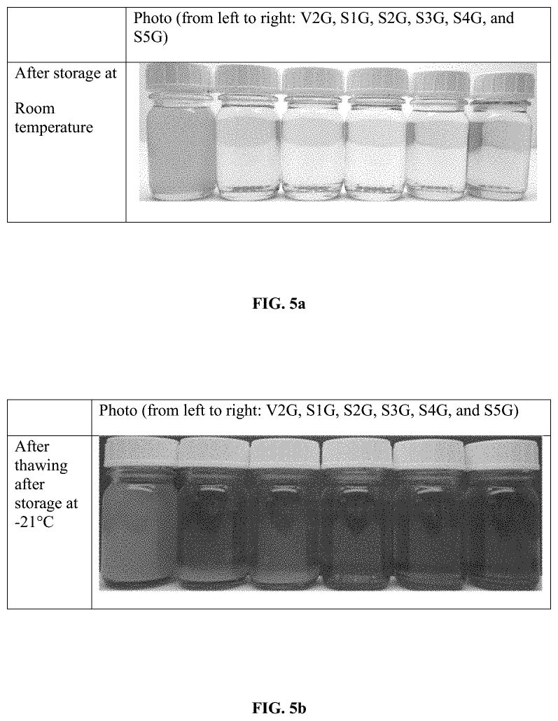

Instead of 1,2-pentanediol, 1,2-hexanediol can also be used. The addition of 1,2-pentanediol leads to better storage stability, and the mixtures S1G to S5G are clear solutions. The mixture without 1,2-pentanediol is already strongly cloudy after a short storage time (cf. FIGS. 5a and 5b).

### Example 6: Determination of the Size of Oil Droplets in an Emulsion According to the Invention

C12/C14 citrate ester mixtures (Table 9Table 9) were prepared and subsequently processed in an O/W emulsion.

The diameters of the oil droplets of the resulting O/W emulsions were measured by laser diffraction.

TABLE 9
Overview of emulsifier mixtures according to the
invention for the measurement of oil droplets.
w/w %

| | A | B | C | D | E |
|---|---|---|---|---|---|
| C12/C14 citrate ester | 100.0 | 60.0 | 60.0 | — | — |
| Caprylic Capric Triglyceride | — | 40.0 | 20.0 | — | 100.0 |
| (INCI) | | | | | |
| 1,2-pentanediol | — | — | 20.0 | 100.0 | — |
| Total | 100.0 | 100.0 | 100.0 | 100.0 | 100.0 |

Preparation of 100 g Batch Size:

C12/C14 citrate ester was heated to 80° C. while stirring and oil and the respective 1,2-alkanediol were added. The resulting solution was stirred for 10 minutes with a blade stirrer at 450 rpm.

Preparation of O/W Emulsions

The following O/W emulsion is prepared with mixtures A, B, C, D, E of Table 10 in the following concentrations (w/w %):

Mixture A: 0.6%

Mixtures B, C, D and E: 1%.

TABLE 10
Compositions of the measured emulsions
w/w %

| | A1 | B1 | C1 | D1 | E1 |
|---|---|---|---|---|---|
| Phase A | | | | | |
| C12/C14 citrate ester | 0.6 | — | — | — | — |
| (Mixture A) | | | | | |
| Citrate ester, Caprylic Capric | — | 1.0 | — | — | — |
| Triglyceride (INCI) | | | | | |
| (Mixture B) | | | | | |
| Citrate ester, Caprylic Capric | — | — | 1.0 | — | — |
| Triglyceride (INCI), 1,2- | | | | | |
| Pentanediol | | | | | |
| (Mixture C) | | | | | |
| 1,2-pentanediol | — | — | — | 1.0 | — |
| (Mixture D) | | | | | |
| Caprylic Capric Triglyceride | — | — | — | — | 1.0 |
| (INCI) | | | | | |
| (Mixture E) | | | | | |
| Cetylstearyl alcohol | 2.0 | 2.0 | 2.0 | 2.0 | 2.0 |
| Glyceryl stearate | 1.5 | 1.5 | 1.5 | 1.5 | 1.5 |
| Cetearyl octanoate | 4.0 | 4.0 | 4.0 | 4.0 | 4.0 |
| Persea gratissima (avocado) | 3.0 | 3.0 | 3.0 | 3.0 | 3.0 |
| oil | | | | | |
| Caprylic Capric Triglyceride | 8.0 | 8.0 | 8.0 | 8.0 | 8.0 |
| (INCI) | | | | | |
| Dimethicone | 0.3 | 0.3 | 0.3 | 0.3 | 0.3 |
| Carbomer | 0.2 | 0.2 | 0.2 | 0.2 | 0.2 |
| Xanthan gum | 0.2 | 0.2 | 0.2 | 0.2 | 0.2 |
| Phase B | | | | | |
| Water (Aqua) | 75.6 | 75.2 | 75.2 | 75.2 | 75.2 |
| 1,2-hexanediol, 1,2-octanediol | 0.5 | 0.5 | 0.5 | 0.5 | 0.5 |
| 4-hydroxyacetophenone | 0.5 | 0.5 | 0.5 | 0.5 | 0.5 |
| Glycerine | 3.0 | 3.0 | 3.0 | 3.0 | 3.0 |
| Phase C | | | | | |
| Water, sodium hydroxide | 0.6 | 0.6 | 0.6 | 0.40 | 0.40 |
| (10% Lsg.) | | | | | |

| | |
|---|---|
| Total | 100.0 |

Preparation of 200 g Batch Size

Phase A (without carbomer and xanthan gum) was heated to 80° C. Carbomer and xanthan gum were then added to phase A. The mixture was then dispersed for 30 seconds with a magnetic stirrer.

Phase B was then slowly added to phase A and emulsified for 3 minutes at 6000 rpm (IKA T25 digital Ultra TURRAX). Phase C was then added while stirring with a blade stirrer for 10 minutes at 100 rpm. With further stirring for 10 minutes at 100 rpm, the obtained emulsion was cooled. Subsequently, the pH was controlled and adjusted to pH 5.7-6.0.

Particle Sizing

The droplet diameter by volume of the O/W emulsions A1, B1, C1 and D1 was then determined by laser diffraction in the Malvern Mastersizer 3000.

The obtained value D_{v0,5} [μm] indicates the volume-related droplet diameter in μm, i.e. a value of 5.8 μm means that 50% of the droplets are smaller than 5.8 μm. The analogous explanation applies to the D_{v0,9} [μm]. A measured value of 11 μm indicates that 90% of the oil droplets have a smaller diameter than 11 μm.

TABLE 11
Results of particle size measurements, emulsions A1 to D1 in μm

| O/W emulsions produced with | D_{v0,5} [μm] | D_{v0,9} [μm] |
|---|---|---|
| 0.6% C12/C14 citrate ester (A) | 5.8 | 11.0 |
| 1% C12/C14 citrate ester Mixture B | 5.2 | 9.3 |
| (0.6% C12/C14 citrate esters, 0.4% | | |
| caprylic capric triglycerides (INCI)) | | |
| 1% C12/C14 citrate ester mixture C | 4.9 | 8.5 |
| (0.6% C12/C14 citrate esters, 0.2% | | |
| caprylic capric triglycerides (INCI), | | |
| 0.2% 1,2-pentanediol) | | |
| 1% 1,2-pentanediol (D) | 14.2 | 38.6 |

Surprisingly, the use of 1% of a mixture of 0.6% C12/C14 citrate ester and 0.4% caprylic capric triglycerides (INCI) resulted in smaller oil droplets compared to 0.6% C12/C14 citrate ester alone.

This means that with the same amount of emulsifier, only the mixture with caprylic capric triglycerides (INCI) achieved a better emulsifying performance, although this oil itself showed no emulsifying effect. A further reduction in the volume-related diameter of the oil droplets was achieved by using 1% of the mixture of 0.6% C12/C14 citrate esters, 0.2% caprylic capric triglycerides (INCI) and 0.2% 1,2-pentanediol.

The use of 1% 1,2-pentanediol alone yielded an emulsion with very large D_{v0,5}-and D_{v0,9} values. Emulsion E1 showed phase separation and could therefore not be measured.

The smaller the volume-related diameter of the oil droplets, the better the emulsifying performance of an emulsifier or emulsifier mixture.

Emulsions with smaller oil droplets exhibit significantly improved physical stability.

### Example 7: Odor Improvement of a Liquid Emulsifier Mixture

Emulsifier mixtures according to the invention were prepared and their odor was investigated after 3 months of storage according to the evaluation key in Table 1 examined.

TABLE 12
Formulations and evaluation after 3 months storage at
40° C. compared to 5° C. sample
w/w %

| | P5 | P6 | P7 | V4 | P8 | P9 | P10 | V5 |
|---|---|---|---|---|---|---|---|---|
| 1,2-pentanediol | 10 | 20 | 30 | 0 | 10 | 20 | 30 | 0 |
| Sunflower oil | 30 | 20 | 10 | 40 | — | — | — | — |
| Rapeseed oil | — | — | — | — | 30 | 20 | 10 | 40 |
| C12/C14 citrate ester | 60 | 60 | 60 | 60 | 60 | 60 | 60 | 60 |
| (based on coconut oil) | | | | | | | | |
| Odor + discoloration | O | O | N | O+ | O | O | N | O+ |

Related to the odor, the mixtures with 30% 1,2-pentanediol (mixture P7 and P10) were judged best, followed by the mixtures with 10% and 20% 1,2-pentanediol (mixtures P5, P8, P6 and P9).

The strongest intrinsic odor was exhibited by the compounds without 1,2-pentanediol (V4 and V5).

### Example 8: Stabilization of Vegetable Oils in Emulsifier Mixtures

Vegetable oils with high contents of unsaturated fatty acids such as rapeseed oil and especially sunflower oil tend to oxidize. To evaluate the effect of a C12/C14 citrate ester on oxidation, the oils were investigated alone as well as in mixtures with C12/C14 citrate esters. For this purpose, sunflower oil and rapeseed oil as well as the respective blends with C12/C14 citrate ester were treated with oxygen and pressure for 48 h at 40° C. and 5 bar in a so-called Oxipres device.

This method is used to determine the shelf life of oils, fats and their blends by treating the samples with oxygen/pressure at a defined temperature. Before and after the Oxipres treatment, the peroxide value was determined, which is a key figure for the assessment of fat deterioration.

The peroxide number is expressed in “milliequivalents of oxygen per kilogram of fat”. The number thus corresponds to the substance quantity of peroxide-bound oxygen atoms in mmol per kilogram of fat (meq O/kg).

TABLE 13
Composition of the mixtures and results of POZ
determinations (before and after Oxipres treatment).
w/w %

| | B1 | B2 | B3 | B4 | B5 |
|---|---|---|---|---|---|
| C12/C14 citrate ester | 100 | — | 50 | — | 50 |
| Rapeseed oil | — | 100 | 50 | — | — |
| Sunflower oil | — | — | — | 100 | 50 |
| Total | 100.0 | | | | |
| POZ before Oxipres (meq O/kg) | 0 | 20 | 10 | 123 | 59 |
| POZ according to Oxipres | 0 | 77 | 11 | 1000 | 18 |
| (meq O/kg) | | | | | |

The highest POZ number after Oxipres treatment was found for sunflower oil (B4; 1000 meq O/kg). The second highest value for rapeseed oil (B2; 77 meq O/kg). The C12/C14 citrate ester shows no peroxides after treatment (B1; 0 meq O/kg).

The blends with C12/C14 citrate esters have a significant stabilizing effect on the vegetable oils and show a significantly lower POZ number compared to the individual oils.

Although the mixtures were stressed by oxygen/pressure and temperature, surprisingly the peroxide number actually decreased compared to the initial value.

### FORMULATION EXAMPLES

### Formulation Example 1: Preferred Emulsifier Mixtures

TABLE 14
Formulations with 1,2-pentanediol and C12/C14 citrate esters

| | A | B | C | D | E | F | G | H |
|---|---|---|---|---|---|---|---|---|
| C12/C14 | 60.0 | 60.0 | 60.0 | 50.0 | 40.0 | 60.0 | 50.0 | 50.0 |
| citrate ester | | | | | | | | |
| Cetearyl | 40.0 | 30.0 | 10.0 | — | — | 30.0 | — | — |
| octanoate | | | | | | | | |
| 1,2-pentanediol | — | 10.0 | 30.0 | 50.0 | — | 10.0 | 30.0 | — |
| Octyldodecanol | — | — | — | — | 60.0 | — | — | — |
| Glycerine | — | — | — | — | — | — | 20.0 | — |
| Isopropyl | — | — | — | — | — | — | — | 50.0 |
| palmitate | | | | | | | | |
| Total | 100.0 | 100.0 | 100.0 | 100.0 | 100.0 | 100.0 | 100.0 | 100.0 |

```
Formulation examples 1 to 15
  1. Day cream O/W
  2: Body lotion
  3: After Sun Balm
  4: Skin soothing body spray
  5: Sunscreen lotion (O/W, broad spectrum protection)
  6: W/O Night Cream
  7: Scalp soothing anti dandruff shampoo
  8: Self tanning cream
  9: Skin protection cream
10: Antiperspirant Roll-On
11: Emulsion with UV-A/B broadband protection
12: Soaking liquid for wet wipes
13: Skin whitening balm with UV-A/UV-B protection
14: Scalp soothing hair conditioner with UV-B/UV-A protection, rinse-off
15: Anti-itch hair conditioner, leave on
```

| | w/w % | | | | | | | | | | | | | | |
|---|---|---|---|---|---|---|---|---|---|---|---|---|---|---|---|
| INCI | 1 | 2 | 3 | 4 | 5 | 6 | 7 | 8 | 9 | 10 | 11 | 12 | 13 | 14 | 15 |
| C12/C14 citrate | | 0.3 | | | | | 0.5 | | 1.0 | | | 1.0 | | | 0.3 |
| ester | | | | | | | | | | | | | | | |
| Mixture P4 | 1.0 | | | 2.0 | | | | 1.5 | | | | | | | |
| (Table 2) | | | | | | | | | | | | | | | |
| Mixture H4 | | | 1.5 | | | | | | | | 1.5 | | | | |
| (Table 5) | | | | | | | | | | | | | | | |
| Mixture O2 | | | | | 1.0 | | | | | | | | | | |
| (Table 6) | | | | | | | | | | | | | | | |
| Mixture P7 | | | | | | 0.5 | | | | 1.0 | | | | | |
| (Table 4) | | | | | | | | | | | | | | | |
| Mixture S4 | | | | | | | | 0.5 | | | | | | 2.0 | |
| (Table 7) | | | | | | | | | | | | | | | |
| Allantoin | | 0.2 | 0.1 | | | | | | 0.2 | | | | | | |
| Water (Aqua), | | | 3.0 | | | 3.0 | 0.4 | | | | | | | | |
| Aloe Barbadensis | | | | | | | | | | | | | | | |
| Leaf Juice | | | | | | | | | | | | | | | |
| Aluminum | | | | | | 1.0 | | | | | | | | | |
| Stearate | | | | | | | | | | | | | | | |
| β-Arbutin | 0.2 | | | | | | | | | | | | | | |
| Butylene glycol | | | 5.0 | | | | | | | | 3.0 | | 3.0 | | |
| Carbomer | | 0.1 | | | 0.2 | | | | | | 0.2 | | | | |
| Cetylhydroxyproline | | 0.1 | | | | | 0.2 | | 0.5 | | | | | | |
| Palmitamide | | | | | | | | | | | | | | | |
| Dicaprylyl ether | | | 4.0 | | | | | | | | | | | | |
| Butyrospermum | | | 1.0 | | | | | | | | | | | | |
| Parkii (Shea | | | | | | | | | | | | | | | |
| Butter) | | | | | | | | | | | | | | | |
| Citric Acid | | 0.4 | | | | | 0.3 | | | | 0.3 | | | | |
| Cocamide MEA | | | | | | | 0.5 | | | | | | | | |
| Climbazole | | | | | | | 0.5 | | | | | | | | |
| Curcuma | | | | | | | | | | 0.5 | | | | | |
| Xanthorrhiza | | | | | | | | | | | | | | | |
| Root Extract | | | | | | | | | | | | | | | |
| Curcuma Longa | | 1.5 | | | | | | | | | | | | | |
| (Turmeric) Root | | | | | | | | | | | | | | | |
| Extract | | | | | | | | | | | | | | | |
| Cetrimonium | | | | | | | | | | | | | | 0.2 | 0.5 |
| Chloride | | | | | | | | | | | | | | | |
| Quaternium-52 | | | | | | | | | | | | | | 0.5 | 4.0 |
| Dihydroxyacetone | | | | | | | | 5.0 | | | | | | | |
| Cyclohexasiloxane | | | | | 2.0 | | | | | | | | | | |
| and | | | | | | | | | | | | | | | |
| Cyclopentasiloxane | | | | | | | | | | | | | | | |
| Cyclomethicone | | | | 0.5 | | | | | | | | | | | |
| Panthenol | | | 1.0 | | | | | | | | | 1.0 | | | |
| Glyceryl Stearate | 1.0 | | | | | | | 1.5 | 1.5 | | | | | 0.5 | 1.0 |
| Citrate | | | | | | | | | | | | | | | |
| Glyceryl Oleate | | | | 2.0 | | | | | | | | | | | |
| Citrate, | | | | | | | | | | | | | | | |
| Caprylic/Capric | | | | | | | | | | | | | | | |
| Triglyceride | | | | | | | | | | | | | | | |
| Water (Aqua), | 0.3 | | | | | | | | | | | | | | |
| Butylene Glycol, | | | | | | | | | | | | | | | |
| Glycerin, Avena | | | | | | | | | | | | | | | |
| Sativa (Oat) | | | | | | | | | | | | | | | |
| Kernel Extract | | | | | | | | | | | | | | | |
| Glycerin, | | | | | | | 2.0 | | | | | | | | |
| Triticum Vulgare | | | | | | | | | | | | | | | |
| (Wheat) Gluten, | | | | | | | | | | | | | | | |
| Water (Aqua) | | | | | | | | | | | | | | | |
| Water (Aqua), | | | | 1.0 | | | | | | | | | | | 2.0 |
| Butylene Glycol, | | | | | | | | | | | | | | | |
| Avena Sativa | | | | | | | | | | | | | | | |
| (Oat) Kernel | | | | | | | | | | | | | | | |
| Extract | | | | | | | | | | | | | | | |
| Polyglyceryl-3 | | | | | | 3.0 | | | | | | | | | |
| Oleate | | | | | | | | | | | | | | | |
| Bisabolol | 0.3 | | | 0.1 | 0.3 | 0.2 | | | | 0.1 | 0.1 | 0.1 | | | |
| Ethylhexyl | | | | | | | | | 2.0 | | | | 0.1 | | |
| ethylisononanoate | | | | | | | | | | | | | | | |
| Disodium EDTA | | | | | 0.1 | | | 0.1 | | | 0.1 | | | 0.1 | |
| Potassium Cetyl | | 1.0 | | | 0.5 | | | | 2.0 | | 1.5 | | 0.1 | | |
| Phosphate, | | | | | | | | | | | | | | | |
| Hydrogenated | | | | | | | | | | | | | | | |
| Palm Glycerides | | | | | | | | | | | | | | | |
| Ethanol | | | | | | | | 2.0 | | 30.0 | | 10.0 | 5.0 | | |
| Glycerin, Water | | 0.3 | | | | | | | 0.5 | | | | | | |
| (Aqua), | | | | | | | | | | | | | | | |
| Rosmarinus | | | | | | | | | | | | | | | |
| officinalis | | | | | | | | | | | | | | | |
| (Rosemary) Leaf | | | | | | | | | | | | | | | |
| Extract | | | | | | | | | | | | | | | |
| Propylene | | | | | | 1.0 | | | | | | | | | |
| Glycol, | | | | | | | | | | | | | | | |
| Hamamelis | | | | | | | | | | | | | | | |
| Virginiana | | | | | | | | | | | | | | | |
| (Witch Hazel) | | | | | | | | | | | | | | | |
| Water, Water | | | | | | | | | | | | | | | |
| (Aqua), | | | | | | | | | | | | | | | |
| Hamamelis | | | | | | | | | | | | | | | |
| Virginiana | | | | | | | | | | | | | | | |
| (Witch Hazel) | | | | | | | | | | | | | | | |
| Extract | | | | | | | | | | | | | | | |
| Famesol | | | | | | | | | | 0.3 | | | | | |
| Perfume oils | 0.3 | 0.5 | 0.1 | 0.2 | 0.4 | 0.4 | 0.5 | 0.3 | 0.3 | 1.0 | 0.1 | 0.5 | 0.4 | 0.5 | 0.1 |
| (Fragrance) PO1, | | | | | | | | | | | | | | | |
| PO2, PO3, PO4, | | | | | | | | | | | | | | | |
| PO5 | | | | | | | | | | | | | | | |
| Menthone | 0.5 | | | | 0.3 | | | | | | | | | | |
| Glycerol Acetal | | | | | | | | | | | | | | | |
| Menthyl Lactate | | | 0.8 | | | | | | | 0.2 | | | | | |
| Menthyl | | | | | | | | | | | 1.0 | | | | |
| Ethylamido | | | | | | | | | | | | | | | |
| Oxalate | | | | | | | | | | | | | | | |
| Sodium Laureth | | | | | | | 37.0 | | | | | | | | |
| Sulfate | | | | | | | | | | | | | | | |
| Glycerin | 3.0 | 2.0 | 4.0 | | 4.7 | 2.0 | | 1.5 | 3.0 | | | | | | |
| Glyceryl stearate | | 2.0 | | | | | | | 2.0 | | 2.0 | | | | |
| Pentylene glycol | | | | 5.0 | | | | 3.5 | | | | | | | |
| Water, Pentylene | | | 1.0 | | | | | | | | | 1.0 | | | |
| Glycol, Glycerin, | | | | | | | | | | | | | | | |
| Fructose, Urea, | | | | | | | | | | | | | | | |
| Citric Acid, | | | | | | | | | | | | | | | |
| Sodium | | | | | | | | | | | | | | | |
| Hydroxide, | | | | | | | | | | | | | | | |
| Maltose, Sodium | | | | | | | | | | | | | | | |
| PCA, Sodium | | | | | | | | | | | | | | | |
| Chloride, | | | | | | | | | | | | | | | |
| Sodium Lactate, | | | | | | | | | | | | | | | |
| Trehalose, | | | | | | | | | | | | | | | |
| Allantoin, | | | | | | | | | | | | | | | |
| Sodium | | | | | | | | | | | | | | | |
| hyaluronate, | | | | | | | | | | | | | | | |
| Glucose | | | | | | | | | | | | | | | |
| Triclosan | | | | | | | | | | 0.3 | | | | | |
| Diisopropyl | | | | | | | | | | | | 0.5 | 1.0 | | |
| adipate | | | | | | | | | | | | | | | |
| Triisononanoin | | 2.0 | | | | | | | 3.0 | | | | 1.0 | | |
| Isopropyl | 4.0 | | | | | | | 4.0 | | | | | | | |
| Palmitate | | | | | | | | | | | | | | | |
| Sorbitol | | | | | | 2.0 | | | | | | | | | |
| Xanthan Gum | 0.2 | 0.1 | | | 0.2 | | | 0.3 | | | 0.2 | | | | |
| Kojic Acid | 1.0 | | | | | | | | | | 0.5 | | | | |
| Cetyl Alcohol | 1.0 | | | | | | | 1.0 | | | 1.2 | | | | |
| Sodium Cetearyl | | | | | | | | | | | 0.7 | | | | |
| Sulfate | | | | | | | | | | | | | | | |
| Cetearyl alcohol | | 3.0 | | | 1.0 | | | | 2.0 | | | | | | |
| Galactoarabinan | | | 0.3 | | | | | | | | | | | 2.5 | 1.5 |
| Magnesium | | | | | | 0.7 | | | | | | | | | |
| chloride | | | | | | | | | | | | | | | |
| Polyquatemium- | | | | | | | 0.5 | | | | | | | | |
| 7 | | | | | | | | | | | | | | | |
| Sodium | | | | | | | | | 0.3 | | | | | | |
| Hydroxide | | | | | | | | | | | | | | | |
| Hydroxyethyl | | | | | | | | | | 0.3 | | | | | |
| cellulose | | | | | | | | | | | | | | | |
| Butyl methoxy- | | | | | 1.0 | | | | | | | | | | |
| dibenzoylmethane | | | | | | | | | | | | | | | |
| Disodium Phenyl | | | | | 10 | | | | | | 22.0 | | 1.5 | | |
| Dibenzimidazole | | | | | | | | | | | | | | | |
| Tetrasulfonate | | | | | | | | | | | | | | | |
| Ethylhexyl | 5.0 | | | | 3.0 | | | | | | | | | | |
| methoxycinnamate | | | | | | | | | | | | | | | |
| Isoamyl p- | | | | | | | | | | | | | 5.0 | | |
| methoxycinnamate | | | | | | | | | | | | | | | |
| Homosalate | | | | | | | | | | | 5.0 | | 5.0 | | |
| Phenylbenzimidazole | | | | | 6.7 | | | | | | | | | | |
| Sulfonic Acid | | | | | | | | | | | | | | | |
| 4-Methylbenzylidene | | | | | 1.5 | | | | | | | | 10.0 | | |
| camphor | | | | | | | | | | | | | | | |
| Ethylhexyl | | | | | 5.0 | | | | | | | | 2.0 | | |
| Salicylate | | | | | | | | | | | | | | | |
| Trideceth-9, | | | | | | | 1.5 | | | | | | | | |
| PEG-5 | | | | | | | | | | | | | | | |
| Ethylhexanoate, | | | | | | | | | | | | | | | |
| Water | | | | | | | | | | | | | | | |
| Caprylic/Capric | 6.0 | | | 4.0 | 2.0 | | | 6.0 | 10.0 | | 2.0 | | | | 1.0 |
| Triglyceride | | | | | | | | | | | | | | | |
| BHT | | | | | | 0.1 | | | | | | | | | |
| Mineral Oil | | | | 4.0 | | | | | | | | | | | |
| Cetearyl | 3.0 | 5.0 | | 7.0 | | 12.0 | | 3.0 | | | 3.0 | | 0.6 | | 0.3 |
| ethylhexoate | | | | | | | | | | | | | | | |
| Stearyl | | 2.0 | | | | | | | | | | | | 3.0 | |
| Heptanoate, | | | | | | | | | | | | | | | |
| Stearyl Caprylate | | | | | | | | | | | | | | | |
| Acrylates/C10- | | | 0.3 | 0.2 | | | | | | | | | | | |
| 30 alkyl acrylate | | | | | | | | | | | | | | | |
| crosspolymer | | | | | | | | | | | | | | | |
| Polyquaternium- | | | | | | | | | | | | | | | 0.1 |
| 10 | | | | | | | | | | | | | | | |
| Propylene glycol | | 5.0 | | | | | | | | | | 3.0 | 0.8 | | 0.8 |
| Retinyl Palmitate | | | | | | 0.2 | | | | | | | | | |
| Polyacrylamide, | | | | | | | | 1.0 | | | | | | | |
| C13-14 | | | | | | | | | | | | | | | |
| Isoparaffin, | | | | | | | | | | | | | | | |
| Laureth-7 | | | | | | | | | | | | | | | |
| Sodium ascorbyl | 2.0 | | 1.0 | | | | | | | | | | | | |
| phosphate | | | | | | | | | | | | | | | |
| Sodium Benzoate | | | | | | | 0.5 | | | | | | | | |
| Sodium Chloride | | | | | | | 1.0 | | | | | | | | |
| Sodium | | 0.3 | 0.6 | 0.4 | | | | | | | 2.8 | | | | |
| Hydroxide | | | | | | | | | | | | | | | |
| PEG-40 | | | | | | | | | | 2.0 | 1.0 | | 2.2 | | |
| Hydrogenated | | | | | | | | | | | | | | | |
| Castor Oil, | | | | | | | | | | | | | | | |
| Trideceth-9, | | | | | | | | | | | | | | | |
| Water (Aqua) | | | | | | | | | | | | | | | |
| Helianthus | | | | | | 5.0 | | | | | | | | | |
| Annuus | | | | | | | | | | | | | | | |
| (Sunflower) Seed | | | | | | | | | | | | | | | |
| Oil | | | | | | | | | | | | | | | |
| Prunus dulcis | | | | | | 5.0 | | | | | | | | | |
| Pentylene | | | 1.0 | | | | | | 1.0 | | | | | | |
| Glycol, Butylene | | | | | | | | | | | | | | | |
| Glycol, | | | | | | | | | | | | | | | |
| Hydroxyphenyl | | | | | | | | | | | | | | | |
| Propamidobenzoic | | | | | | | | | | | | | | | |
| Acid | | | | | | | | | | | | | | | |
| 2-methyl 5- | | | | | | | | | | 0.5 | | | | | |
| cyclohexylpentanol | | | | | | | | | | | | | | | |
| Dimethyl | | | | 0.5 | | | | | | | | | | | |
| phenylbutanol | | | | | | | | | | | | | | | |
| 1.2-hexanediol, | | | | | | | | | | | 0.5 | | | | |
| caprylylglycol, | | | | | | | | | | | | | | | |
| 1,2-hexanediol, | 0.5 | | | | | | | | | | | | | | |
| caprylylglycol, | | | | | | | | | | | | | | | |
| tropolone | | | | | | | | | | | | | | | |
| Maltodextrin, | | 0.1 | | | 0.3 | 1.0 | | | | | | | | | |
| Rubus Fruticosus | | | | | | | | | | | | | | | |
| (Blackberry) | | | | | | | | | | | | | | | |
| Leaf Extract | | | | | | | | | | | | | | | |
| Cetearyl | | | | | | | | | | | 1.5 | | | | |
| Nonanoate | | | | | | | | | | | | | | | |
| Phenoxyethanol, | | | | | | | 1.0 | | | | | | | | |
| Decylene Glycol, | | | | | | | | | | | | | | | |
| 1,2 Hexanediol | | | | | | | | | | | | | | | |
| Laureth-9 | | 0.5 | | | | | | | | | | | | 1.0 | |
| Bisabolol, | | | | 0.1 | | | | | | | | | | | |
| Zingiber | | | | | | | | | | | | | | | |
| Officinale | | | | | | | | | | | | | | | |
| (Ginger) Root | | | | | | | | | | | | | | | |
| Extract | | | | | | | | | | | | | | | |
| Hexyldecanol, | | | | | | | | | 2.0 | | | | | | |
| Bisabolol, | | | | | | | | | | | | | | | |
| Cetylhydroxyproline | | | | | | | | | | | | | | | |
| Palmitamide, | | | | | | | | | | | | | | | |
| Stearic Acid, | | | | | | | | | | | | | | | |
| Brassica | | | | | | | | | | | | | | | |
| Campestris | | | | | | | | | | | | | | | |
| (Rapeseed) | | | | | | | | | | | | | | | |
| Sterols | | | | | | | | | | | | | | | |
| Pentylene glycol, | | | 1.5 | | | | | | | | 0.5 | | | | |
| 4-t- | | | | | | | | | | | | | | | |
| butylcyclohexanol | | | | | | | | | | | | | | | |
| Water, Pentylene | | | | | | | | | | | | | | | 1.5 |
| Glycol, Sodium | | | | | | | | | | | | | | | |
| Lauryl | | | | | | | | | | | | | | | |
| Sulfoacetate, | | | | | | | | | | | | | | | |
| Sodium Oleoyl | | | | | | | | | | | | | | | |
| Sarcosinate, | | | | | | | | | | | | | | | |
| Sodium | | | | | | | | | | | | | | | |
| Chloride, | | | | | | | | | | | | | | | |
| Disodium | | | | | | | | | | | | | | | |
| Sulfoacetate, | | | | | | | | | | | | | | | |
| Sodium Oleate, | | | | | | | | | | | | | | | |
| Sodium Sulfate | | | | | | | | | | | | | | | |
| Zingiber | 0.1 | | | | | 0.1 | | | | | | | | | |
| Officinale | | | | | | | | | | | | | | | |
| (Ginger) Root | | | | | | | | | | | | | | | |
| Extract | | | | | | | | | | | | | | | |
| Phenylethyl | 0.5 | | | | | | | | | | 0.5 | | 1.0 | | |
| resorcinol | | | | | | | | | | | | | | | |
| Cocamidopropyl | | | | | | | 6.0 | | | | | 1.0 | 1.0 | | |
| Betaine | | | | | | | | | | | | | | | |
| Polyglyceryl 3- | | | | | | | | | 0.3 | | | | | | |
| Caprate | | | | | | | | | | | | | | | |
| C12-15 alkyl | | | 5.0 | | 5.0 | | | | | | | | | | |
| benzoate | | | | | | | | | | | | | | | |
| Sodium Laureth | | | | | | | | | | | | | 4.0 | | |
| Sulfate | | | | | | | | | | | | | | | |
| Tocopheryl | | | 0.5 | | 0.5 | 3.0 | | | 0.3 | | 0.5 | | | | |
| Acetate | | | | | | | | | | | | | | | |
| Triethanolamine | | | | | 0.5 | | | | | | | | 0.5 | | |
| Water (Aqua) | ad 100 | | | | | | | | | | | | | | |
| Aluminum | | | | | | | | | | 37 | | | | | |
| Zirconium | | | | | | | | | | | | | | | |
| Pentachlorohydrate | | | | | | | | | | | | | | | |
| (40% aqueous | | | | | | | | | | | | | | | |
| solution) | | | | | | | | | | | | | | | |

Formulations of perfume oils P01, P02, P03, P04 and P05

| | |
|---|---|
| Ingredient (INCI) | w/w % |

P01

| | |
|---|---|
| ALDEHYDE C14 SO-CALLED | 2 |
| ALLYL AMYL GLYCOLATE 10% | 5 |
| DPG | |
| ANISIC ALDEHYDE PURE | 5 |
| APPLE OLIFFAC TYPE | 10 |
| BENZYLACETATE | 50 |
| BERGAMOT IDENTOIL ® | 15 |
| COLORLESS | |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL | 2 |
| BUTYRATE | |
| DIPROPYLENE GLYCOL | 178 |
| EBANOL | 2 |
| ETHYL DECADIENOATE TRANS | 2 |
| CIS-2,4 10% IPM | |
| FLOROSA | 5 |
| FRAMBINON ® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 | 1 |
| 10% DPG | |
| HEXENYL SALICYLATE CIS-3 | 5 |
| HEXYL CINNAMIC ALDEHYDE | 70 |
| ALPHA | |
| HEXYL SALICYLATE | 50 |
| HYDROXY CITRONELLAL | 10 |
| ISO E SUPER | 15 |
| ISORALDINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL | 5 |
| 10% DPG | |
| TONALIDE | 7 |
| VERTOCITRAL 10% DPG | 5 |
| VERTOFIX | 15 |

P02

| | |
|---|---|
| Acetophenones, 10% in DPG | 10 |
| n-Undecanal | 5 |
| Aldehydes C14, so-called (peach | 15 |
| aldehyde) | |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyl oxides | 5 |
| Eucalyptol | 10 |
| Geraniol | 40 |
| Nerol | 20 |
| Geranium oil | 15 |
| Hexenol cis-3, 10% in DPG | 5 |
| Hexenyl salicylate cis-3 | 20 |
| Indole, 10% in DPG | 10 |
| Alpha-ionone | 15 |
| Beta-ionone | 5 |
| Lilial ® (2-methyl-3-(4-tert-butyl- | 60 |
| phenyl)propanal) | |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |

P03

| | |
|---|---|
| Benzyl acetate | 60 |
| Citronellyl acetate | 60 |
| Cyclamenaldehydes (2-methyl-3-(4- | 20 |
| isopropylphenyl)propanal | |
| Dipropylene glycol (DPG) | 60 |
| Ethyllinalool | 40 |
| Florol (2-isobutyl-4- | 30 |
| methyltetrahydro- | |
| 2H-pyran-4-ol) | |
| Globanone ® [(E/Z)-8- | 180 |
| cyclohexadecen-1-one] | |
| Hedione ® | 140 |
| (methyldihydrojasmonate) | |
| Hexenyl salicylate, cis-3 | 10 |
| Vertocitral (2,4-dimethyl-3- | 5 |
| cyclohexenecarboxaldehydes) | |
| Hydratropaldehydes, 10% in DPG | 5 |
| Isodamascone (1-(2,4,4-trimethyl-2- | 5 |
| cyclohexen-1-yl)-2-buten-1-one, | |
| 10% in DPG | |
| Isomuscone (cyclohexadecanone) | 40 |
| Jacinthaflor (2-methyl-4-phenyl- | 10 |
| 1,3-dioxolane) | |
| Cis-jasmone, 10% in DPG | 20 |
| Linalool | 50 |
| Linalyl acetate | 30 |
| Methyl benzoate, 10% in DPG | 25 |
| para-Methyl cresol, 10% in DPG | 10 |
| Nerol | 20 |
| Phenylpropylaldehydes | 5 |
| 2-Phenylethyl alcohol | 82 |
| Tetrahydrogeraniol | 13 |
| 2,2-Dimethyl-3-cyclohexyl-1- | 80 |
| propanol | |

P04

| | |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. bergapten-free | 60 |
| CALONE ® 1951 10% in DPG | 15 |
| COUMARIN | 5 |
| CYCLOGALBANATE ® 10% in | 10 |
| DPG | |
| ALPHA-DAMAS CONE 1% in DPG | 20 |
| DIHYDROMYRCENOL | 10 |
| ETHYL LINALOOL | 75 |
| ETHYL LINALYL ACETATE | 50 |
| ETHYL MALTOL 1% in DEP | 10 |
| ETHYLENE BRASSYLATE | 80 |
| (MACRO) | |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYL SALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |

P05

| | |
|---|---|
| AMAROCITE ® | 10 |
| AMBROCENIDE ® 10% in DPG | 5 |
| AMBROXIDE | 15 |
| AURELIONE ® (7/8- | 70 |
| cyclohexadecenone) (MACRO). | |
| BERGAMOT OIL. bergapten-free | 90 |
| CALONE ® 1951 10% in DPG | 20 |
| CARAWAY OIL | 10 |
| CITRAL | 20 |
| COUMARIN | 10 |
| ALPHA-DAMAS CONE 1% in DPG | 15 |
| DIHYDROMYRCENOL | 70 |
| ESTRAGON OIL | 10 |
| ETHYL LINALOOL | 100 |
| ETHYL LINALYL ACETATE | 90 |
| EUGENOL | 10 |
| EVERNYL ® | 5 |
| FRUCTATE ® | 5 |
| GERANIUM OIL | 5 |
| HEDIONE ® HC/30 | 100 |
| HELIONAL ® | 10 |
| INDOLE 10% in DPG | 5 |
| ISO E SUPER ® | 100 |
| KEPHALIS ® | 5 |
| LAVENDER OIL | 40 |
| CITRUS OIL | 80 |
| LILIAL ® | 30 |
| MANDARIN OIL | 20 |
| MUSCENONE (MACRO) | 5 |
| SANDRANOL ® | 10 |
| VANILLIN 10% in DPG | 5 |
| DIPROPYLENE GLYCOL | 30 |

1-15. (canceled)

16. An emulsifier mixture comprising:
(a) a mixture of glycerol esters comprising at least one compound of formula (I) and/or a salt thereof: 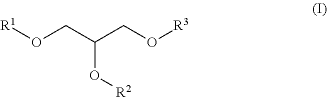
wherein
(i) at least one of R¹, R² and R³ is a citric acid residue attached by an ester bond; and
(ii) at least one of R¹, R², and R³ is a fatty acid residue attached by an ester bond; and
wherein more than 55 wt. % of the one or more compounds of formula (I) comprise at least one fatty acid residue independently selected from caproic acid, caprylic acid, capric acid, lauric acid, and myristic acid, based on a total weight of the one or more compounds of formula (I); and/or
more than 50 wt. % of the one or more compounds of formula (I) comprise at least one fatty acid residue independently selected from lauric acid and myristic acid, based on the total weight of the compounds of formula (I); and/or
less than 40 wt. % of the one or more compounds of formula (I) comprise at least one fatty acid residue independently selected from palmitic acid, palmitoleic acid, stearic acid, oleic acid, linoleic acid, and linolenic acid, based on the total weight of the one or more compounds of formula (I); and
(b) at least one 1,2-alkanediol, 1,3-alkanediol, 1,4-alkanediol, 1,5-alkanediol, or a mixture thereof; and

17. The emulsifier mixture of claim 16, wherein
more than 50 wt. % of the one or more compounds of formula (I) comprise at least one fatty acid residue independently selected from lauric acid and myristic acid, based on the total weight of the compounds of formula (I); and
less than 40 wt. % of the one or more compounds of formula (I) comprise at least one fatty acid residue independently selected from palmitic acid, palmitoleic acid, stearic acid, oleic acid, linoleic acid, and linolenic acid.

18. The emulsifier mixture of claim 16, wherein the 1,2-alkanediol, 1,3-alkanediol, 1,4-alkanediol, and the 1,5-alkanediol are selected from C5-C12 alkanediols.

19. The emulsifier mixture of claim 18, wherein the C5-C12 alkanediols are selected from 1,2-pentanediol, 1,2-hexanediol, 1,2-heptanediol, 1,2-octanediol, 1,2- nonanediol, 1,2-decanediol, 1,2-dodecanediol, 1,3-propanediol, 1,4-butanediol, 1,5-pentanediol, and mixtures thereof.

20. The emulsifier mixture of claim 16 comprising:
(a) 10 to 98 wt. % of the mixture of glycerol esters; and
(b) 1 to 90 wt. % of the least one 1,2-alkanediol, 1,3-alkanediol, 1,4-alkanediol, 1,5-alkanediol, or a mixture thereof;
based on a total weight of the emulsifier mixture.

21. The emulsifier mixture of claim 16 comprising:
(a) 20 to 80 wt. % of the mixture of glycerol esters; and
(b) 2 to 75 wt. % of the at least one 1,2-alkanediol, 1,3-alkanediol, 1,4-alkanediol, 1,5-alkanediol, or a mixture thereof;
based on a total weight of the emulsifier mixture.

22. The emulsifier mixture of claim 16, further comprising:
(c) 1 to 20 wt.-% of 4-hydroxyacetophenone;
based on a total weight of the emulsifier mixture.

23. The emulsifier mixture of claim 16 comprising:
(b) 5 to 10 wt. % of at least one 1,2-alkanediol; and
(c) 1 to 20 wt. % of 4-hydroxyacetophenone;
based on a total weight of the emulsifier mixture.

24. The emulsifier mixture of claim 16, wherein the at least one 1,2-alkanediol comprises 1,2-octanediol.

25. The emulsifier mixture of claim 16, wherein the at least one fatty acid residue is obtained from a fatty acid-containing reactant, wherein the highest fatty acid content of the reactant in % by weight based on a total fatty acid content is from C8 to C18 fatty acids.

26. The emulsifier mixture of claim 16, wherein the at least one fatty acid residue is of natural, biotechnological, or chemical origin.

27. The emulsifier mixture of claim 25, wherein the fatty acid-containing reactant is selected from coconut oil, babassu oil, sunflower oil, rapeseed oil, neutral oil, palm kernel oil, macúba oil, microalgae oil, and mixtures thereof.

28. The emulsifier mixture of claim 16, wherein the emulsifier mixture is a liquid and optionally comprises at least one lipophilic solvent.

29. The emulsifier mixture of claim 28 comprising the at least one lipophilic solvent, wherein the at least one lipophilic solvent is selected from Cocos nucifera (Coconut) Oil, Orbignya oleifera Seed Oil, Helianthus annuus (Sunflower) Seed Oil, Olea europaea (Olive) Fruit Oil, Brassica campestris (Rapeseed) Seed Oil, Caprylic Capric Triglycerides, Prunus amygdalus dulcis (Sweet Almond) Oil, Simmondsia chinensis (Jojoba) Seed Oil, Elaeis guineensis (Palm) Kernel Oil, Acrocomia aculeata Kernel Oil, Squalane, Cetearyl Nonanoate, Propanediol Dicaprylate/Caprate, Cetearyl Nonanoate, Diisopropyl Adipate, Ethylhexyl Stearate, Butyrospermum parkii (Shea) Butter, Microalgae Oil, Ricinus communis (Castor) Seed Oil, Squalane, Triethyl Citrate, Polyglyceryl-4 Caprate, Polyglyceryl-2 Caprate, Polyglyceryl-3 Caprylate, Polyglyceryl-3 Caprate, Coco-Caprylate/Caprate, Isoamyl Laurate, Cetearyl Alcohol, Polyglyceryl 4-Cocoate, Glyceryl Caprylate, Glyceryl Caprylate/Caprate, Glyceryl Undecylenate, Glyceryl Laurate, Glyceryl Oleate, Glyceryl Stearate, Dicaprylyl Ether, Hexyldecanol, Octyldodecanol, C15-19 Alkanes, Dicaprylyl Carbonate, Oleyl Erucate, Glyceryl Isostearate, and mixtures thereof.

30. The emulsifier mixture of claim 16, wherein the emulsifier mixture exhibits a homogeneous phase transition upon thawing.

31. The emulsifier mixture of claim 16, wherein the emulsifier mixture is a clear solution.

32. A method for forming an oil-in-water emulsion comprising combining the emulsifier mixture of claim 16 with oil and water and emulsifying the combination.

33. A method for improving stability of an oil-in-water emulsion comprising incorporating the emulsifier mixture of claim 16 into the oil-in-water emulsion.

34. A oil-in-water emulsion comprising:
(i) the emulsifier mixture of claim 16;
(ii) an oil phase;
(iii) an aqueous phase; and
(iv) optionally, at least one compound to lower or increase the viscosity of the emulsion.

35. A cleaning, cosmetic, or pharmaceutical preparation, or an edible preparation serving for nutrition or pleasure comprising the emulsifier mixture of claim 16.
